(19) United States
(12) Patent Application Publication
(10) Pub. No.: US 2007/0244200 A1
(43) Pub. Date: Oct. 18, 2007

(54) Method for triggering angiogenic response

(76) Inventor: Somnath Mukhopadhyay, Durham, NC (US)

Correspondence Address: CHARLES EDISON SMITH, 1512 S. ALSTON AVE., DURHAM, NC, 27707, US

(21) Appl. No.: 11/404,456

(22) Filed: Apr. 15, 2006

(51) Int. Cl. A61K 31/16

(52) U.S. Cl. 514/627

(57) ABSTRACT

The use of known chemical anadamide compounds and its analog methanandamide as a molecule to evoke angiogenic responses as measured by the increase in cord formation and increase in branching points or cord junction numbers A rabbit aortic endothelial cell is used as a model and in in vitro cord formation assay.

### FIELD OF THE INVENTION

This invention pertains to the field of utilization of drug chemical compounds to evoke new blood vessel formation and lengthening of blood vessels.

### BACKGROUND OF THE INVENTION

There are sources in the literature that pertain to administration of pharmacologic chemical compounds and methods describing various many laboratories use thereof for assessing the in vitro angiogenesis assay. Some references in the literature for employing these techniques to a certain degree, each of which are hereby incorporated herein by reference, include: Shailubhai K, Dheer S, Picker D, Kaur G, Sausville E A, Jacob G S. Atiprimod is an inhibitor of cancer cell proliferation and angiogenesis. J Exp Ther Oncol. 4: 267-79, 2004; Soriano J V, Liu N, Gao Y, Yao Z J, Ishibashi T, Underhill C, Burke T R Jr, Bottaro D P. Inhibition of angiogenesis by growth factor receptor bound protein 2-Src homology 2 domain bound antagonists. Mol Cancer Ther. 3:1289-99, 2004; Malinda, K M, Nomizu M, Chung M, et al. Identification of laminin α1 and β1 chain peptides active for endothelial cell adhesion, tube formation, and aortic sprouting. FASEB J 13: 53-62, 1999; and Grant D S, Kinsella J L, Fridman R, et al. Interaction of endothelial cells with a laminin A chain peptide (SIKVAV) in vitro and induction of angiogenic behavior in vivo. J Cell Physiol 53: 614-25, 1992.

Moore, U.S. Pat. No. 6,916,852, is hereby incorporated herein by reference, discloses a method of administering pharmacologic chemical compounds for regulation of the tone of small blood vessels.

### DESCRIPTION OF THE FIGURES

FIG. 1 is a graph showing the response of blood vessels to application of anadamide and methanandamide chemical compounds.

### DESCRIPTION OF THE INVENTION

The known chemical entity anadamide and its analog, methanandamide as a molecule, to evoke formation of cords of blood vessels in subjects. This invention relates to the use of known endogenous cannabinoid compound anadamide (arachidonyl ethanolamide) and its analog methanandamide (methyl arachidonyl ethanolamide) for promoting angiogenic response under in vitro and ex vivo conditions. More particularly, the invention relates to a method using endogenous cannabinoid compound anadamide (arachidonyl ethanolamide) and its analog methanandamide (methyl arachidonyl ethanolamide) to demonstrate that anadamide and methanandamide can stimulate formation of tubes and branching of those tubes from endothelial cells under in vitro conditions. The present invention also illustrates that the molecules of anadamide and methanandamide promotes sprouting or blood capillary formation under ex vivo conditions. The present invention further illustrates use of anadamide and methanandamide compounds for treating of wounds, wound healing and the formation of new blood vessels during stroke related blockage of blood capillaries.

### EXAMPLE

### Cord Formation on Matrigel

### 96 Well Plate

In carrying this invention, materials needed in this example include commercially available items as follows: a 96 Well Plate, for example, flat bottom 96 well plate (Falcon 353072); Media-BM-2 (Clonetics CC-3162), DMEM (Gibco Cat#11960044); Matrigel-Beckton Dickinson (Cat#40234); Cells; HUVEC (2×10⁶/ml) in EBM-2 in EBM-2; and Image Analysis-Image-Pro Plus In this example, the assay is performed under hood is, a Laminar Flow Hood. Frozen Matrigel (Beckton Dickinson 40234) is placed overnight on ice to thaw. Then, a concentration of matrigel is adjusted to 10 mg/ml using DMEM media. An ice cold 96 well plate is then placed on ice in a flat dish. Using an ice cold tip, gently layer 60 μl of matrigel (10 mg/ml; kept on ice) in each well. Gently tap the plate so that matrigel is evenly distributed in each well. The assay thereafter involves the following steps and protocols of: Removing the ice under the plate and incubate the plate for 15 min at room temperature; transfer the plate to a 37° C. incubator for at least 30 min for the Matrigel to polymerize; prepare methanandamide at 2X the desired concentration in culture media; harvest RAEC (rabbit aortic endothelial cells) in culture media and adjust the cell density to 2×10⁵/ml; mix 500 μl of drug with 500 μl of cell suspension and place 200 μl of this suspension in duplicate wells on polymerized matrigel (being cautious to not touch matrigel with the tip and to dispense cells very gently and slowly on the matrigel to avoid damage to matrigel layer); incubate the compound concentration in a 37° C. humidified incubator for 18-24 h observe condition of cords under a microscope.; take at least three pictures (10 X) from different fields from each well (or six pictures from duplicate wells); count number of junctions and tube length (mm) from each picture using Image Pro Image Analysis System; and analyze the data.

In accordance with a preferred embodiment of the present invention, the effect of methanandamide on rabbit aortic endothelial cells (RAEC) was tested for its ability to induce cord formation on matrigel. This process entails an accepted in vitro assay for angiogenesis. The procedures involve analyses with three different concentrations of methanandamide compound and repeated the experiments three different times in duplicate. As mentioned earlier above in the protocol, the 3 pictures from each well were taken. Therefore, for each concentration (data point) we have 18 scores. The analysis entails the measure of two parameters: (1) cord length; and (2) cord junction number.

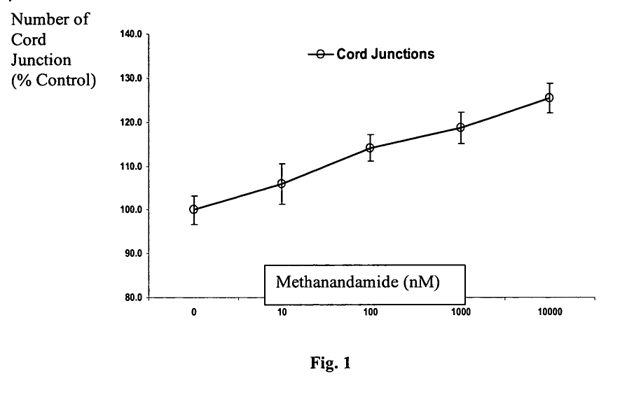

Reference is made generally to Table 1 for the quantification of cord length in conjunction with FIG. 1 for Cord junction number for illustrating the results of one study of angiogenic response in blood vessels of a subject.

TABLE I

| | Percent Increase in Cord length | | | |
|---|---|---|---|---|
| Methanandamide (nM) | Set I | Set II | Set III | Mean |
| 10 | 12, 11, | 16, 21, | 14, 10, | 14 |
| | 9, 13, | 14, 18, | 17, 13, | |
| | 12, 10 | 13, 12 | 15, 17 | |
| 50 | 25, 32, | 22, 28, | 30, 30, | 25 |
| | 32, 19 | 22, 18, | 26, 35, | |
| | 17, 22 | 17, 26 | 34, 25 | |
| 100 | 45, 42 | 37, 41, | 38, 42, | 40 |
| | 39, 47, | 44, 32, | 44, 47, | |
| | 42, 40 | 34, 44 | 36, 34 | |

In FIG. 1, there is shown representations for eighteen (18) scores of data points for each concentration of methanandamide compound analyzed. The scores indicated for each concentration of methanandamide resulted from three different concentrations of methanandamide, i.e. 10 nM, 50 nM, and 100 nM. Three (3) pictures taken from each field or well (Six (6) pictures for duplicate wells). Each treatment or analysis was done in duplicate in 3 separate sets, i.e., Set I, Set II, and Set III. Therefore, for each concentration data point has eighteen (18) scores (3×2×3=18 data points).

The foregoing example and analysis describes the use of the known chemical compounds, anadamide and its analog methanandamide as a molecule that can evoke or trigger lengthening blood vessels or new blood vessel formation.

What is claimed is:

1. A method of causing an angiogenic response of cell blood vessels comprising administering to a non-mammal subject a sufficient quantity of molecules of a anadamide compound.

2. The method of claim 1 wherein said angiogenic response is an increase of blood vessels is measured by cord formations.

3. The method of claim 2 wherein said non-mammal is a rabbit and the cell blood vessels is the aortic endothelial cell of said rabbit.

4. The method of claim 2 wherein said angiogenic response is an increase of blood vessels as measured by cord junction numbers.

5. A method of causing an angiogenic response of cell blood vessels comprising administering to a non-mammal subject a sufficient quantity of molecules of a methanandamide compound.

6. The method of claim 5 wherein said angiogenic response is an increase of blood vessels as measured by cord formations.

7. The method of claim 6 wherein said non-mammal is a rabbit and the cell blood vessels is the aortic endothelial cell of said rabbit.

8. The method of claim 7 wherein said angiogenic response is an increase of blood vessels as measured by cord junction numbers.

9. A method of causing an angiogenic response of cell blood vessels comprising
administering a sufficient quantity of molecules of synthetic compound of a endogenous bioactive lipid anadamide, said cell blood vessels is a rabbit aortic endothelial cell as a model and in vitro cord formation assay.

10. The method of claim 9 wherein angiogenic responses is an increase as measured by the increase in cord formation and an increase in cord junction number

11. A method of causing an angiogenic response of cell blood vessels comprising
administering a sufficient quantity of molecules of synthetic compound of an endogenous bioactive lipid methanandamide, said cell blood vessels is a rabbit aortic endothelial cell as a model and in in vitro cord formation assay.

12. The method of claim 11 wherein angiogenic responses is an increase as measured by the increase in cord formation and an increase in cord junction number

13. The method of claim 12 wherein the said methanandamide is a synthetic compound and analog of endogenous bioactive lipid anadamide (arachidonyl ethanolamide) evokes angiogenic responses as measured by the increase in cord formation and increase in branching point (cord junction number) using rabbit aortic endothelial cell as a model and in in vitro cord formation assay.
